(19) United States
(12) Patent Application Publication
(10) Pub. No.: US 2021/0162134 A1
(43) Pub. Date: Jun. 3, 2021

(54) MICROSYRINGE UNIT

(71) Applicants: KYOTO UNIVERSITY, Kyoto-shi, Kyoto (JP); KABUSHIKI KAISHA TOP, Tokyo (JP)

(72) Inventors: Jun Takahashi, Kyoto (JP); Daisuke Doi, Kyoto (JP); Tetsuhiro Kikuchi, Kyoto (JP); Ken Kato, Tokyo (JP)

(21) Appl. No.: 17/262,944

(22) PCT Filed: Jul. 26, 2019
(86) PCT No.: PCT/JP2019/029532

§ 371 Date: Jan. 25, 2021

(30) Foreign Application Priority Data
Jul. 30, 2018 (JP) 2018-142134

(51) Int. Cl. A61M 5/315

(52) U.S. Cl. CPC A61M 5/31531; A61M 2005/311

(57) ABSTRACT

Provided is a microsyringe unit capable of improving the accuracy of the injection position of liquid in a brain. The microsyringe unit of the present invention is composed of a microsyringe (100) and a needle guide (200). The microsyringe (100) includes a cylindrical needle (110), a plunger (120) that is passed through the needle (110), and a needle base portion (130) that supports the needle (110). The needle base portion (130) abuts on a guide base portion (230) of the needle guide (200), thereby causing a part of the needle (110) to protrude from the tip of the needle guide (200) in a reference state in which forward movement of the needle (110) passed through the needle guide (200) is stopped.

### TECHNICAL FIELD

The present invention relates to a microsyringe unit including a microsyringe and a needle guide.

### BACKGROUND ART

There has been proposed a microsyringe for injecting a liquid such as a drug or a tissue piece or therapeutic cell preparation composition into a target area of a patient's brain (refer to Patent Literature 1). With the tip of a cylindrical needle in the liquid such as a drug, the plunger that is passed through the inside of the needle moves backward from the tip of the needle, by which the liquid is inhaled inside the needle. With the needle that is passed through the needle guide constituting the needle guide whose tip is inserted into the patient's brain, the plunger passed through the needle moves forward, by which the liquid is ejected from the tip of the needle.

### CITATION LIST

### Patent Literature

Patent Literature 1: Japanese Utility Model Application Laid-Open No. S51-44389

### SUMMARY OF INVENTION

### Technical Problem

If, however, the liquid ejected from the tip of the needle comes into contact with the tip of the needle guide, the liquid is more likely to spread outward in the radial direction of the needle accordingly, by which it may be difficult to accurately eject the liquid in front of the needle and thus to accurately inject the liquid into the target area in the brain.

Therefore, it is an object of the present invention to provide a microsyringe unit capable of improving the accuracy of the injection position of liquid in a brain.

### Solution to Problem

The present invention relates to a microsyringe unit composed of a microsyringe that includes a cylindrical needle and a plunger that is passed through the needle, and a cylindrical needle guide.

According to an aspect of the present invention, there is provided a microsyringe unit wherein the base portion of the needle abuts on a guide base portion that supports the needle guide, thereby causing the tip of the needle to protrude from the tip of the needle guide in a first specified state in which forward movement of the needle passed through the needle guide is stopped.

According to the microsyringe unit having the above configuration, the tip of the needle is separated from the tip of the needle guide in the first specified state, thereby ensuring that the liquid such as a drug ejected from the tip of the needle does not come into contact with the tip of the needle guide to spread in the radial direction of the needle. This allows the liquid ejected from the tip of the needle to be accurately ejected in front of the needle and thus the liquid to be accurately injected into a target area in a brain.

In the microsyringe unit of the present invention, preferably the tip of the needle has a convex curved surface whose outer diameter gradually decreases toward the tip.

According to the microsyringe unit having the above configuration, a gap is secured between the convex curved surface at the tip of the needle and a tissue of the brain, thereby enabling the liquid ejected from the tip of the needle to be retained in the gap to suppress the radial spread of the liquid. This further ensures that the liquid does not come into contact with the tip of the needle guide to spread in the radial direction of the needle.

In the microsyringe unit of the present invention, preferably the surface roughness of the convex curved surface of the tip of the needle is greater than the surface roughness of the outer side surface of the needle.

According to the microsyringe unit having the above configuration, the tip of the needle is hydrophilic, thereby enabling the wetness of the outer side surface of the tip to be lower than the wetness of the convex curved surface, the liquid ejected from the tip of the needle to be retained in the gap between the convex curved surface of the tip of the needle and the tissue of the brain, and the liquid to be prevented from spreading radially along the outer side surface of the needle. This more reliably prevents the liquid from coming into contact with the tip of the needle guide and spreading in the radial direction of the needle.

In the microsyringe unit of the present invention, preferably the tip of the needle guide has a convex curved surface whose outer diameter gradually decreases toward the tip, and the radius of curvature of the convex curved surface of the tip of the needle is greater than the radius of curvature of the convex curved surface of the tip of the needle guide.

According to the microsyringe unit having the above configuration, a large gap is secured between the convex curved surface of the tip of the needle and the tissue of the brain due to the radius of curvature of the convex curved surface of the tip of the needle greater than the radius of curvature of the convex curved surface of the tip of the needle guide accordingly, thereby enabling more liquid to be retained in the gap to prevent radial spread of the liquid. Moreover, the radius of curvature of the convex curved surface of the tip of the needle guide is smaller than the radius of curvature of the convex curved surface of the tip of the needle, thereby reliably preventing the tip of the needle guide from damaging the brain tissue accordingly.

In the microsyringe unit of the present invention, preferably the microsyringe unit further includes a stylet that is passed through the needle guide and is columnar at least in the tip, wherein the base portion of the stylet abuts on the guide base portion that supports the needle guide, thereby causing the tip of the stylet to protrude from the tip of the needle guide in a second specified state in which forward movement of the stylet passed through the needle guide is stopped.

According to the microsyringe unit having the above configuration, the tip of the stylet protruding from the needle guide that is fixed while entering the patient's brain deviates the tissue of the brain in the second specified state, thereby enabling formation of a space for the tip of the needle protruding from the needle guide to enter in the first specified state that is implemented after the second specified state.

In the microsyringe unit of the present invention, preferably the tip of the stylet has a convex curved surface whose outer diameter gradually decreases toward the tip and the radius of curvature of the convex curved surface of the tip of the stylet is greater than the radius of curvature of the convex curved surface of the tip of the needle guide.

According to the microsyringe unit having the above configuration, the radius of curvature of the convex curved surface of the tip of the stylet is greater than the radius of curvature of the convex curved surface of the tip of the needle guide, thereby preventing the tissue of the brain from being damaged accordingly with the brain deviated by the tip of the stylet protruding from the needle guide that is fixed while entering the patient's brain in the second specified state.

In the microsyringe unit of the present invention, preferably the protruding length of the stylet from the tip of the needle guide in the second specified state is equal to or greater than the protruding length of the needle from the tip of the needle guide in the first specified state. Thereby, a cell injection space can be secured since a space is formed in the brain for the volume caused by a difference in protruding length between the stylet and the needle.

According to the microsyringe unit having the above configuration, the tip of the stylet deviates the brain's tissue as described above, thereby enabling a sufficient space for the tip of the needle to be formed.

In the microsyringe unit of the present invention, preferably the tip of the plunger has a convex curved surface whose outer diameter gradually decreases toward the tip and the radius of curvature of the convex curved surface of the tip of the plunger is smaller than the radius of curvature of the convex curved surface of the tip of the needle.

According to the microsyringe unit having the above configuration, the radius of curvature of the convex curved surface of the tip of the plunger is smaller than the radius of curvature of the convex curved surface of the tip of the needle, thereby enabling a gap between the tip of the plunger and the inner side surface of the needle to be reduced accordingly with the tip position of the plunger matching the tip position of the needle. Therefore, when the plunger is pushed in until the tip position of the plunger matches the tip position of the needle, the amount of liquid that stays in the gap is reduced and thus the amount of liquid ejected from the tip of the needle can be accurately adjusted.

In the microsyringe unit of the present invention, preferably at least a part of the needle is composed of a transparent member.

According to the microsyringe unit having the above configuration, when the plunger passed through the needle whose tip is in contact with the liquid moves backward, whether the liquid has been properly contained in the internal space of the needle can be seen from the part composed of a transparent member of the needle.

In the microsyringe unit of the present invention, preferably the microsyringe unit further includes a cylindrical packing that is arranged so as to abut on the side wall of a through hole penetrating the base portion of the needle and having a narrow portion that narrows toward the tip inside the through hole and so as to abut on the narrow portion at the tip and that the plunger is passed through.

According to the microsyringe unit having the above configuration, the liquid can be prevented from entering the space on the rear side of the packing in the internal space of the needle.

In the microsyringe unit of the present invention, preferably the needle guide has at least one stepped portion where its outer diameter decreases discontinuously from the rear end side to the tip side.

Thereby, in the needle guide, the outer diameter of the tip side part that is thinner than the rear end side part across the stepped portion and is continuous with the tip can be designed within an appropriate numerical range from the viewpoint of being inserted into the brain.

### BRIEF DESCRIPTION OF DRAWINGS

FIG. 1 is an explanatory diagram related to the configuration of a microsyringe unit as an embodiment of the present invention.

FIG. 2 is an enlarged explanatory diagram of a part A in FIG. 1.

FIG. 3 is an enlarged explanatory diagram of a part B in FIG. 1.

FIG. 4 is an enlarged explanatory diagram of a part C in FIG. 1.

FIG. 5 is an explanatory diagram related to the configuration of a syringe outer cylinder and a plunger guide.

FIG. 6 is an explanatory diagram related to the configuration of a plunger and a syringe inner cylinder.

FIG. 7 is an explanatory diagram related to the configuration of a stylet.

FIG. 8 is an explanatory diagram related to a combination usage of the stylet and the needle guide.

FIG. 9 is an enlarged explanatory diagram of a part D in FIG. 8.

FIG. 10 is an enlarged explanatory diagram of a part E in FIG. 8.

### DESCRIPTION OF EMBODIMENTS

(Configuration)

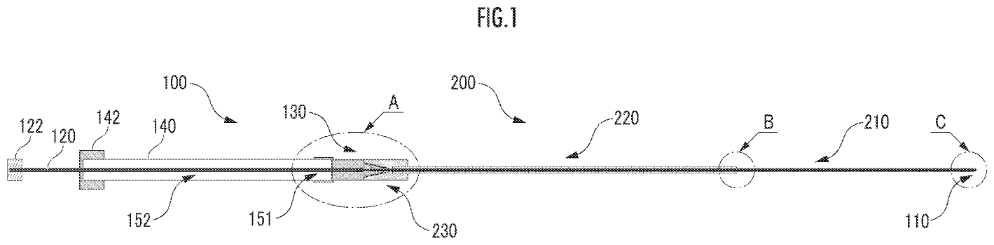

A microsyringe unit as an embodiment of the present invention, which is illustrated in its entirety in FIG. 1 and whose essential parts are respectively illustrated in FIGS. 2 to 4, is composed of a microsyringe 100 and a needle guide 200. For description, the right side of FIGS. 1 to 4 is defined as the tip side or front side of the microsyringe 100 and of the needle guide 200, and the left side is defined as the rear end side or rear side of the microsyringe 100 and of the needle guide 200.

As illustrated in FIG. 1, the microsyringe 100 includes a needle 110, a plunger 120, a needle base portion 130, a syringe outer cylinder 140, an outer cylinder 151, and a syringe inner cylinder 152. Each of the needle 110, the plunger 120, the needle base portion 130, the outer cylinder 151, and the syringe inner cylinder 152 is made of metal such as stainless steel. The syringe outer cylinder 140 is made of transparent glass, synthetic resin, or the like.

As illustrated in FIG. 2, the needle base portion 130 is composed of a first base element 131, a second base element 132, and a third base element 133. The first base element 131 is a substantially truncated cone-like part extending along the central axis line and having a through hole whose diameter is substantially equal to or slightly smaller than the outer diameter of the needle 110. The second base element 132 is a substantially cylindrical part having an inner diameter that is greater than the diameter of the through hole of the first base element 131 and having an outer diameter that is locally expanded at the rear end. The third base element 133 is a substantially cylindrical part having an inner diameter that is substantially equal to or greater than the outer diameter of the second base element 132 and having an outer diameter that is substantially equal to the rear end of the second base element 132. The first base element 131, the second base element 132, and the third base element 133 are arranged coaxially and formed integrally so that the tip of the second base element 132 is continuous with the rear end (or lower bottom) of the first base element 131 and the tip of the third base element 133 is continuous with the enlarged diameter portion at the rear end of the second base element 132.

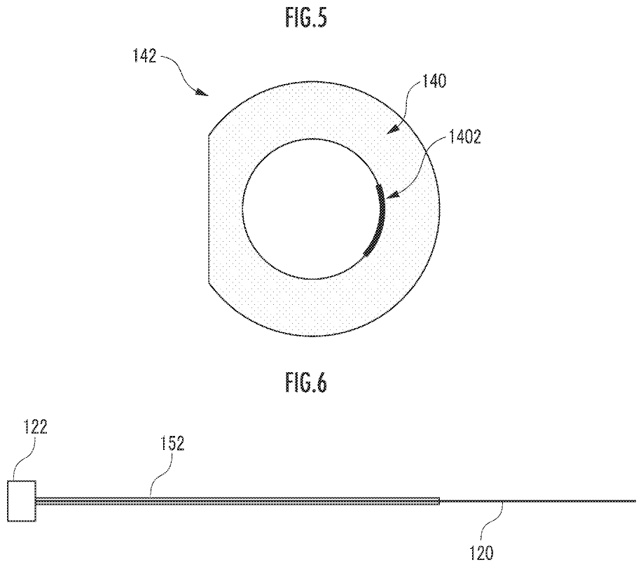

As illustrated in FIG. 1, the needle 110 has a substantially cylindrical shape or has a cross section that is formed in a substantially annular linear shape, and is fixed to the needle base portion 130 with its rear end inserted into the through hole of the first base element 131. As illustrated in FIG. 4, the tip of the needle 110 may be R-processed so that the outer diameter of the tip of the needle 110 gradually decreases or becomes thinner toward the tip, thereby forming a convex curved surface (R surface). The radius of curvature R₁₁₀ of the convex curved surface of the tip of the needle 110 is designed to be, for example, within the range of R0.05 to 0.15 mm.

The surface roughness of the convex curved surface of the tip of the needle 110 may be designed to be greater than the surface roughness of the outer side surface of the needle 110. For example, the surface roughness Ra of the convex curved surface of the tip of the needle 110 is designed to be within the range of 1.0 to 6.0 μm, preferably 1.0 to 4.0 μm, and more preferably 1.0 to 2.0 μm, while the surface roughness Ra of the outer side surface of the tip of the needle 110 is designed to be within the range of 0.04 to 1.0 μm, preferably 0.04 to 0.50 μm, and more preferably 0.04 to 0.10 μm. In this case, it is preferable to make the tip of the needle 110 hydrophilic. For example, the tip of the needle 110 can be made hydrophilic by imparting a hydrophilic functional group to the needle 110 made of SUS or of synthetic resin by plasma treatment or the like.

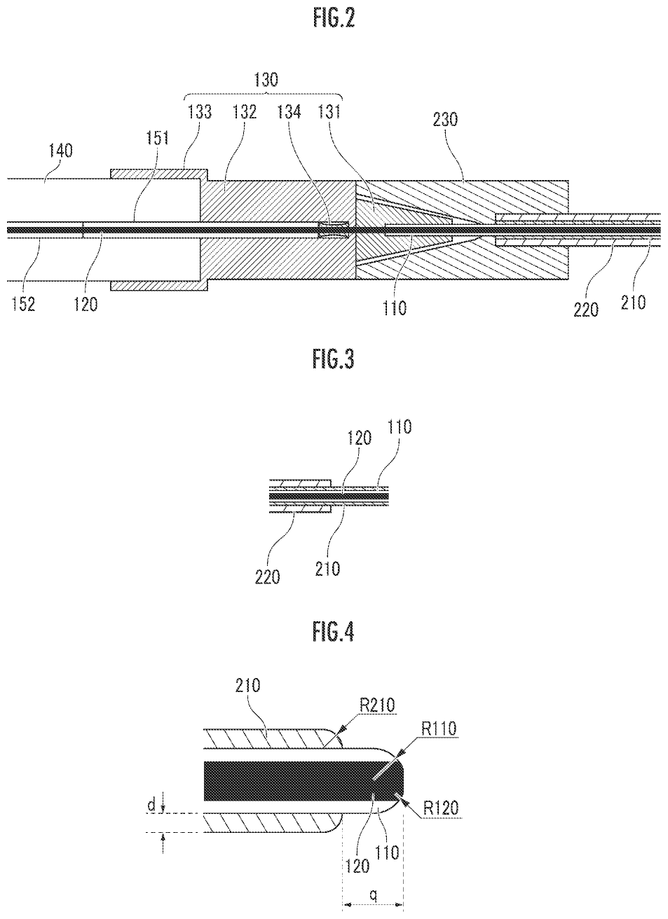

As illustrated in FIG. 3, the plunger 120 has a diameter slightly smaller than the inner diameter of the needle 110 and is formed in a substantially columnar shape longer than the needle 110 or in a linear shape with a substantially circular cross section, and the plunger 120 is passed through the internal space of the needle 110. As illustrated in FIG. 4, the tip of the plunger 120 may be processed (R-processed) so that the diameter of the plunger 120 gradually decreases as it approaches the tip to form a convex curved surface (R surface). The radius of curvature R₁₂₀ of the convex curved surface of the tip of the plunger 120 is designed to be, for example, within the range of R0 to 0.15 mm. A substantially columnar plunger holder 122 is attached to the rear end of the plunger 120.

As illustrated in FIG. 2, the syringe outer cylinder 140 is made of substantially cylindrical transparent glass, synthetic resin, or the like having an outer diameter substantially equal to the inner diameter of the third base element 133 of the needle base portion 130. Similarly, as illustrated in FIG. 2, the inner diameter of the syringe outer cylinder 140 is substantially equal to the inner diameter of the second syringe base portion, and the syringe outer cylinder 140 is fixed to the needle base portion 130 with the tip inserted into the third base element 133 of the needle base portion 130. As illustrated in FIG. 1, a substantially flat-shaped plunger guide 142 having a through hole is fixed to the rear end of the syringe outer cylinder 140.

As illustrated in FIG. 5, the plunger guide 142 is formed in a shape in which the outer part of a chord (a shape like the alphabetical letter “D”) of a circle is cut out from the circle when facing the microsyringe 100 in the front-back direction. On the outer peripheral surface of the syringe outer cylinder 140, a scale 1402 may be provided to indicate the amount of liquid inhaled by the needle 110 (or the amount of advance and retreat of the plunger 120 with respect to the needle 110) in the outer peripheral surface region corresponding to the side opposite to the chord with respect to the center of the circle in the cross section of the plunger guide 142 or in the outer peripheral surface region deviated in the circumferential direction therefrom.

As illustrated in FIG. 2, the outer cylinder 151 is formed in a substantially cylindrical shape having an inner diameter greater than the diameter of the plunger 120 and having an outer diameter substantially equal to the inner diameter of the second base element 132 of the needle base portion 130. Also as illustrated in FIG. 2, the outer cylinder 151 is fixed to the needle base portion 130 in a state where the plunger 120 penetrates the internal space of the outer cylinder 151 and a tip-side part of the outer cylinder 151 is partially passed through the internal space of the second base element 132 of the needle base portion 130.

As illustrated in FIG. 2, in the internal space of the second base element 132 of the needle base portion 130, there is provided a syringe packing 134 that abuts on the step between the tip of the outer cylinder 151 and the through hole of the first base element 131. As illustrated in FIG. 2, the syringe packing 134 is made of a cylindrical and flexible material such as synthetic resin that has an outer peripheral surface shaped so that the diameter gradually decreases from the tip to the center and then gradually increases from the center to the rear end or has a drum-shaped outer peripheral surface and that has an inner diameter substantially equal to the diameter of the plunger 120.

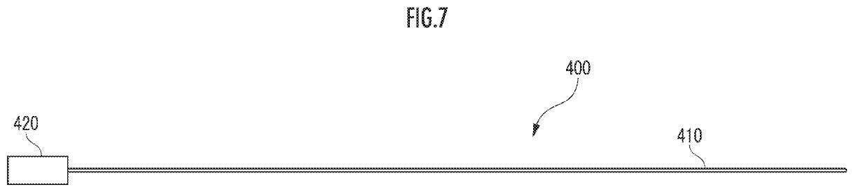

The syringe inner cylinder 152 is formed in a substantially cylindrical shape having an inner diameter that is substantially equal to or slightly greater than the diameter of the plunger 120 and having an outer diameter that is substantially equal to the through hole of the plunger guide 142 (and smaller than the outer diameter of the outer cylinder 151). As illustrated in FIG. 6, the syringe inner cylinder 152 is fixed to the plunger holder 122 at the rear end by the plunger 120 penetrating its internal space on the rear side of the outer cylinder 151. As illustrated in FIG. 1, the syringe inner cylinder 152 penetrates the through hole of the plunger guide 142. Therefore, when the plunger holder 122 moves forward and backward relative to the syringe outer cylinder 140, the plunger 120 and the syringe inner cylinder 152 move forward and backward integrally through the through hole of the plunger guide 142 (see FIGS. 1 and 6).

As illustrated in FIG. 2, with the tip of the syringe inner cylinder 152 and the rear end of the outer cylinder 151 in contact with each other, the tip of the needle 110 and the tip of the plunger 120 are aligned in the same position in their axial directions.

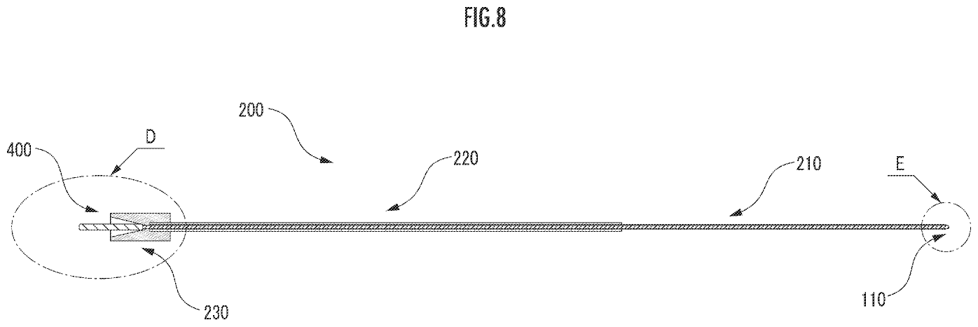

As illustrated in FIG. 8, the needle guide 200 is composed of an inner needle guide 210 and an outer needle guide 220 and is supported by a guide base portion 230. The inner needle guide 210, the outer needle guide 220, and the guide base portion 230 are each made of metal such as stainless steel. At least one of the inner needle guide 210, the outer needle guide 220, and the guide base portion 230 may be made of thermosetting resin.

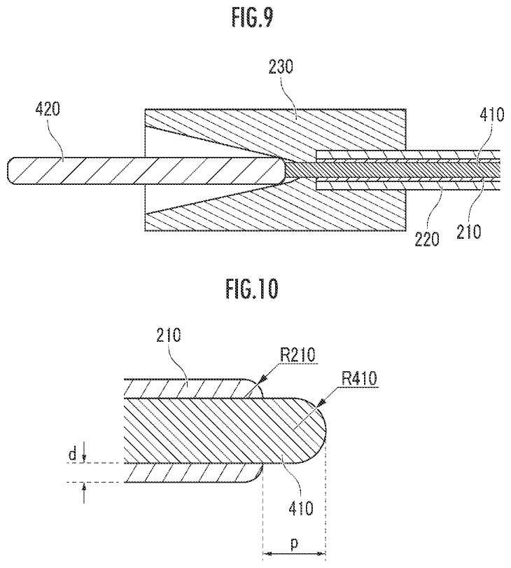

As illustrated in FIG. 9, the guide base portion 230 is formed in a substantially cylindrical shape in which an internal space on the front side is formed in a substantially columnar shape and an internal space on the rear side contiguous thereto is formed in a substantially truncated cone-like shape. As also illustrated in FIG. 9, the internal space on the rear side of the guide base portion 230 has an upper bottom with a smaller diameter than the internal space on the front side and than the upper bottom of the first base element 131 of the needle base portion 130, has a lower bottom with a greater diameter than the lower bottom of the first base element 131, and has an inner side surface shape that follows the outer side surface shape of the first base element 131.

As illustrated in FIG. 9, the inner needle guide 210 is formed in a substantially cylindrical shape having an outer diameter smaller than the diameter of the internal space on the front side of the guide base portion 230 and having an inner diameter substantially equal to the outer diameter of the needle 110. As illustrated in FIG. 10, the tip of the inner needle guide 210 may be processed (R-processed) so that the outer diameter of the inner needle guide 210 gradually decreases as it approaches the tip to form the convex curved surface (R surface) (see FIG. 4). The radius of curvature R₂₁₀ of the convex curved surface of the tip of the inner needle guide 210 is designed to be, for example, within the range of R0.05 to 0.15 mm.

There may be a magnitude relation between the radius of curvature R₁₁₀ of the convex curved surface of the tip of the needle 110, the radius of curvature R₁₂₀ of the convex curved surface of the tip of the plunger 120, and the radius of curvature R₂₁₀ of the convex curved surface of the tip of the inner needle guide 210 as represented by the relational expression (1).

R₁₂₀<R₂₁₀<R₁₁₀ (1)

As illustrated in FIG. 9, the outer needle guide 220 is formed in a substantially cylindrical shape, which is shorter in the axial direction than the inner needle guide 210, having an outer diameter substantially equal to the diameter of the internal space on the front side of the guide base portion 230 and having an inner diameter substantially equal to the outer diameter of the inner needle guide 210. Therefore, the needle guide 200 has a step in the outer diameter as illustrated in the part B of FIG. 1 and in FIG. 3. The outer needle guide 220 may be processed (R-processed) so that the outer diameter gradually decreases or becomes thinner as approaching the tip in the tip part.

Each of the inner needle guide 210 and the outer needle guide 220 has a wall thickness that ensures enough strength to prevent bending thereof as it is longer in the axial direction. In the case where each of the inner needle guide 210 and the outer needle guide 220 is made of, for example, stainless steel, its wall thickness is designed to be, for example, within the range of 0.1 to 1.0 mm, preferably 0.1 to 0.5 mm, and more preferably 0.2 to 0.5 mm.

As illustrated in FIG. 9, the inner needle guide 210 is fixed to the outer needle guide 220 in a state that the rear side part of the inner needle guide 210 is passed through the internal space of the outer needle guide 220 so that the rear end of the inner needle guide 210 is located at the same position as the rear end of the outer needle guide 220 in the axial direction. Also illustrated in FIG. 9, the outer needle guide 220 is fixed to the guide base portion 230 with its rear end passed through the internal space on the front side of the guide base portion 230.

The microsyringe unit as an embodiment of the present invention may further include a stylet 400 illustrated in FIG. 7. As illustrated in FIG. 7, the stylet 400 includes a substantially columnar shaft 410 that is passed through the inner needle guide 210 and a substantially columnar base portion 420 that has a diameter greater than the diameter of the shaft 410 and supports the shaft 410. As illustrated in FIG. 8, the stylet 400 is passed through the needle guide 200.

As illustrated in FIG. 9, the base portion 420 of the stylet 400 abuts on the guide base portion 230 supporting the needle guide 200, thereby stopping forward movement of the stylet 400 passed through the needle guide 210 and of its shaft 410. In this state (second specified state), the tip of the shaft 410 of the stylet 400 protrudes from the tip of the needle guide 200 as illustrated in FIG. 10. As illustrated in FIG. 10, the tip of the shaft 410 of the stylet 400 may have a convex curved surface (R surface) such that the outer diameter gradually decreases toward the tip (see FIG. 10). There may be a magnitude relation represented by a relational expression (2) between the radius of curvature R₄₁₀ of the convex curved surface of the tip of the shaft 410 of the stylet 400 and the radius of curvature R₂₁₀ of the convex curved surface of the tip of the inner needle guide 210.

R₂₁₀<R₄₁₀ (2)

(Functions)

At the time of brain surgery, the needle guide 200 in the second specified state is inserted into a brain, first. At this time, the tip position and posture of the needle guide 200 are determined by the needle guide 200 supported by a support mechanism (not illustrated). In the “second specified state,” the substantially columnar stylet 400 is passed through the internal space of the needle guide 200 (that is, the inner needle guide 210), and the base portion 420 of the stylet 400 abuts on the guide base portion 230, by which forward movement of the stylet 400 is stopped (see FIGS. 8 and 9). In the second specified state, the tip of the shaft 410 of the stylet 400 protrudes from the tip of the needle guide 200 by a protruding length p (see FIG. 10), and the tip of the shaft 410 of the stylet 400 deviates a tissue of the patient's brain, by which a minute space is secured in the brain by that amount. The stylet 400 is then pulled out of the needle guide 200 supported by the support mechanism.

Subsequently, the needle 110 of the microsyringe 100 with a liquid containing a therapeutic composition inhaled at the tip in advance is passed through the needle guide 200 supported by the support mechanism. The substantially truncated cone-like lateral inner side surface that defines the internal space on the rear side of the guide base portion 230 constitutes a tapered surface that approaches the central axis line of the guide base portion 230 as it goes forward in the longitudinal section (See FIG. 2). Due to this tapered surface, the needle 110 is smoothly guided to the internal space of the inner needle guide 200, and the first base element 131 of the needle base portion 130 is smoothly guided to the internal space on the rear side of the guide base portion 230.

The needle base portion 130 and the guide base portion 230 are moved relative to each other so that they are closer to each other with the needle 110 passed through the needle guide 200, by which the tip of the second base element 132 of the needle base portion 130 abuts on the rear end of the guide base portion 230 (see FIG. 2). In addition thereto, the outer side surface of the first base element 131 of the needle base portion 130 may abut on the inner side surface of the internal space of the rear side of the guide base portion 230. Thereby, there is implemented a “first specified state” in which forward movement of the needle 110, which is passed through the needle guide 200, is stopped (see FIG. 1).

In the first specified state, a part of the needle 110 protrudes from the tip of the needle guide 200 (that is, the inner needle guide 210) (see FIG. 4), and then the needle 110 enters the minute space in the brain secured by the tip of the shaft 410 of the stylet 400 as described above. At this time, the plunger 120 is located behind the position illustrated in FIG. 4, and the internal space of the needle 110 is filled with a liquid such as a cell preparation composition on the tip side of the plunger 120.

The protruding length q of the needle 110 is equal to or similar to the protruding length p of the shaft 410 of the stylet 400. The protruding length q of the needle 110 is within the range of 1d to 30d, for example, with respect to the maximum wall thickness d of the inner needle guide 210, and when d=0.15 mm, q=0.15 to 4.5 mm. The protruding length q of the needle 110 is preferably within the range of 2d to 15d, more preferably 5 to 10d. The protruding length p of the shaft 410 of the stylet 400 is within the range of 1d to 30d, for example, with respect to the maximum wall thickness d of the inner needle guide 210, and when d=0.15 mm, q=0.15 to 4.5 mm. The protruding length p of the stylet 400 is preferably within the range of 3d to 16d, more preferably 6 to 11d.

When the plunger holder 122 moves forward relative to the syringe outer cylinder 140 in this state, the plunger 120 moves forward and a liquid such as a cell preparation composition is injected into the minute space in the brain from the opening at the tip of the needle 110 (see FIG. 4). Since the tip of the needle 110 is separated from the tip of the needle guide 200 in the first specified state (see FIG. 4), a liquid such as a cell preparation composition ejected from the tip of the needle 110 comes into contact with the tip of the needle guide 200 (that is, the inner needle guide 210), thereby reliably preventing the situation in which the liquid spreads in the radial direction of the needle 110. This allows the liquid ejected from the tip of the needle 110 to be accurately ejected in front of the needle 110 and thus the liquid to be accurately injected into a target area in the brain.

(Other Embodiments of the Present Invention)

At least a part of the needle 110, especially the tip thereof, may be formed of an acrylic or other transparent member. Thereby, when the plunger 120 inserted into the needle 110 whose tip is in contact with the liquid moves backward, whether the liquid has been properly contained in the internal space of the needle 110 can be seen from the part composed of the transparent member of the needle 110.

### DESCRIPTION OF REFERENCE NUMERALS

100 Microsyringe
110 Needle
120 Plunger
122 Plunger holder
130 Needle base portion
134 Syringe packing
140 Syringe outer cylinder
142 Plunger guide
151 Outer cylinder
152 Syringe inner cylinder
200 Needle guide
210 Inner needle guide
220 Outer needle guide
230 Guide base portion
400 Stylet
410 Shaft
420 Stylet base portion

1. A microsyringe unit comprising:
a microsyringe including a cylindrical needle and a plunger that is passed through the needle; and
a cylindrical needle guide that the needle is passed through,
wherein a base portion of the needle abuts on a guide base portion that supports the needle guide, thereby causing a tip of the needle to protrude from a tip of the needle guide in a first specified state in which forward movement of the needle passed through the needle guide is stopped.

2. The microsyringe unit according to claim 1, wherein the tip of the needle has a convex curved surface whose outer diameter gradually decreases toward a tip end of the needle.

3. The microsyringe unit according to claim 2, wherein a surface roughness of the convex curved surface of the tip of the needle is greater than a surface roughness of an outer side surface of the needle.

4. The microsyringe unit according to claim 2, wherein:
the tip of the needle guide has a convex curved surface whose outer diameter gradually decreases toward a tip end of the needle guide; and
a radius of curvature of the convex curved surface of the tip of the needle is greater than a radius of curvature of the convex curved surface of the tip of the needle guide.

5. The microsyringe unit according to claim 1, further comprising a stylet that is passed through the needle guide and is columnar at least in a tip of the stylet,
wherein a base portion of the stylet abuts on the guide base portion that supports the needle guide, thereby causing the tip of the stylet to protrude from the tip of the needle guide in a second specified state in which forward movement of the stylet passed through the needle guide is stopped.

6. The microsyringe unit according to claim 5, wherein:
the tip of the stylet has a convex curved surface whose outer diameter gradually decreases toward a tip end of the stylet; and
a radius of curvature of the convex curved surface of the tip of the stylet is greater than a radius of curvature of the convex curved surface of the tip of the needle guide.

7. The microsyringe unit according to claim 5, wherein a protruding length of the stylet from the tip of the needle guide in the second specified state is equal to or greater than a protruding length of the needle from the tip of the needle guide in the first specified state.

8. The microsyringe unit according to claim 1, wherein:
the tip of the plunger has a convex curved surface whose outer diameter gradually decreases toward a tip end of the plunger; and
a radius of curvature of the convex curved surface of the tip of the plunger is smaller than a radius of curvature of the convex curved surface of the tip of the needle.

9. The microsyringe unit according to claim 1, wherein at least a part of the needle is composed of a transparent member.

10. The microsyringe unit according to claim 1, further comprising a cylindrical packing that is arranged so as to abut on a side wall of a through hole penetrating a base portion of the needle and having a narrow portion that narrows toward a tip end inside the through hole and so as to abut on the narrow portion at the tip and that the plunger is passed through.

11. The microsyringe unit according to claim 1, wherein the needle guide has at least one stepped portion where its outer diameter decreases discontinuously from a rear end side to a tip side.
